(19) United States
(12) Patent Application Publication
(10) Pub. No.: US 2015/0297306 A1
(43) Pub. Date: Oct. 22, 2015

(54) MANAGING A DETACHABLE COMPONENT OF A MEDICAL DEVICE

(71) Applicants: Avi LAZAR, Ra'ananna (IL); Zachi PELES, Tel Aviv (IL); Roni BIBI, Ra'anana (IL); Adam LAPRAD, San Francisco, CA (US); Yoni DAGAN, Ra'anana (IL); PULMONE ADVANCED MEDICAL DEVICES, LTD., Ra-anana (IL)

(72) Inventors: Avi Lazar, Ra'anana (IL); Zachi Peles, Tel Aviv (IL); Roni Bibi, Tel Mond (IL); Adam Laprad, San Francisco, CA (US); Yoni Dagan, (IL)

(73) Assignee: PulmOne Advanced Medical Devices, Ltd., Ra'anana (IL)

(21) Appl. No.: 14/648,122

(22) PCT Filed: Nov. 27, 2013
(86) PCT No.: PCT/US13/72371

§ 371 Date: May 28, 2015

Related U.S. Application Data
(60) Provisional application No. 61/731,029, filed on Nov. 29, 2012.

(51) Int. Cl. A61B 19/00; A61B 5/097; A61M 16/10; A61B 5/091; A61B 5/00; A61M 16/12; A61M 16/00; A61B 5/087; A61B 5/08

(52) U.S. Cl. CPC A61B 19/44; A61B 5/087; A61B 5/097; A61M 16/1055; A61B 5/0806; A61B 5/0004; A61M 16/12; A61M 16/0006; A61B 5/091; A61B 2019/448; A61M 2202/025; A61M 2202/0266; A61B 2560/0443

(57) ABSTRACT

A pulmonary function testing (PFT) system includes a PFT device operable to receive a respiratory airflow from a patient through an airflow chamber; a mouthpiece coupled to the PFT device and in airflow communication with the airflow chamber, the mouthpiece including a bacterial filter and associated with a unique identifier and patient data; and a control system communicably coupled with the mouthpiece and the PFT device. The control system is configured to perform operations including interpreting at least one of the unique identifier or the patient data associated with the mouthpiece; and based on the interpretation, adjusting a status of the PFT device to determine a pulmonary function parameter.

### CROSS REFERENCE TO RELATED APPLICATION

This application claims priority to U.S. Provisional Patent Application Ser. No. 61/731,029, filed Nov. 29, 2012, and entitled “Method and System for Identifying Use of a Detachable Piece Connected to a Medical Device,” the entire contents of which are hereby incorporated by reference.

### TECHNICAL BACKGROUND

The present disclosure relates to monitoring uses of medical devices, and more particularly, but not exclusively, to monitoring use of a detachable part of a medical device by utilizing identification technologies.

### BACKGROUND

Large and expensive medical devices are often used to test or diagnose more than one person—often dozens of people a day—leading the medical community to be widely concerned with the possibility of contamination of medical devices and/or of transferring of diseases between users of a medical device. As a result, many medical devices include detachable parts, intended to be used for a single patient, and then discarded. For example, needles used to draw blood, surgical staplers, and trocars are all designed to be used for a single subject and to be immediately disposed of subsequent to use.

However, lack of sensitivity, frugality, or industrial competition can lead medical providers or their personnel to ignore the consequences of reuse or sharing of a detachable part of a medical device for multiple uses. It is often very difficult, for patients as well as for doctors, to identify whether an item has been previously used by a patient and is now being reused for another patient or for the same patient, or if the detachable part is indeed original and was just removed from its packaging.

In the context of pulmonary function testing devices, a subject being tested breathes into a filter by placing his or her mouth around a mouthpiece of the filter and breathing into the filter. As such, the filter is exposed to the subject's body fluids such as saliva and phlegm, and to any bacterial or viral infections the subject may be carrying. However, because such filters do not engage blood, because the classic pulmonary diseases, such as emphysema, COPD, and asthma, are not contagious, and because medical providers test dozens of subjects a day, medical providers may stray from good practice and allow more than one user to use a single filter. Alternatively, the medical provider may operate the device without any filter whatsoever, compromising the sterility of the device itself. Operating a pulmonary function testing device in either of these manners is in contradiction to medical recommendations and guidelines of leading institutions in this field, such as the American Thoracic Society, which require a filter to be used on every device, a filter to be changed between use of the device for different individual users, and a new filter to be used at each doctor's visit even for a single user.

### SUMMARY

The present disclosure relates to the field of monitoring use of medical devices, and more particularly, but not exclusively, to methods for monitoring use of a detachable part of a medical device by utilizing identification technologies.

In a general implementation, a pulmonary function testing (PFT) system includes a PFT device operable to receive a respiratory airflow from a patient through an airflow chamber; a mouthpiece coupled to the PFT device and in airflow communication with the airflow chamber, the mouthpiece including a bacterial filter and associated with a unique identifier and patient data; and a control system communicably coupled with the mouthpiece and the PFT device. The control system is configured to perform operations including interpreting at least one of the unique identifier or the patient data associated with the mouthpiece; and based on the interpretation, adjusting a status of the PFT device to determine a pulmonary function parameter.

Other general implementations include computer-implemented methods and computer-readable media. For example, a system of one or more computers can be configured to perform particular actions by virtue of having software, firmware, hardware, or a combination of them installed on the system that in operation causes or cause the system to perform the actions. One or more computer programs can be configured to perform particular actions by virtue of including instructions that, when executed by data processing apparatus, cause the apparatus to perform the actions.

In a first aspect combinable with any of the general implementations the control system includes an identification module of the PFT device, the identification module including a processor communicably coupled with the PFT device and the mouthpiece; and a controller communicably coupled with the PFT device and the mouthpiece.

In a second aspect combinable with any of the previous aspects, the processor of the identification module is operable to interpret at least one of the unique identifier or the patient data associated with the mouthpiece, and the controller is operable to adjust the status of the PFT device based on the interpretation to determine a pulmonary function parameter.

In a third aspect combinable with any of the previous aspects, the operation of adjusting a status of the PFT device to determine a pulmonary function parameter includes enabling the PFT device to determine the pulmonary function parameter.

In a fourth aspect combinable with any of the previous aspects, the operation of adjusting a status of the PFT device to determine a pulmonary function parameter includes disabling the PFT device to determine the pulmonary function parameter.

In a fifth aspect combinable with any of the previous aspects, the control system is further operable to perform operations including providing a notification to an operator of the PFT device of the disabling of the PFT device to determine the pulmonary function parameter.

In a sixth aspect combinable with any of the previous aspects, the notification includes an error message.

In a seventh aspect combinable with any of the previous aspects, the control system is further operable to perform operations including, based on disabling the PFT device to determine the pulmonary function parameter, invalidating an identification marker that includes at least one of the unique identifier or the patient data.

In an eighth aspect combinable with any of the previous aspects, invalidating an identification marker that includes at least one of the unique identifier or the patient data includes at least one of erasing at least one of the unique identifier or the patient data; changing the unique identifier; or adjusting a validity flag of the identification module.

In a ninth aspect combinable with any of the previous aspects, the PFT device includes a hand-held PFT device.

In a tenth aspect combinable with any of the previous aspects, the PFT includes one of a spirometer, a plethysmograph, a diffusion capacity device, a helium dilution device, a nitrogen wash-out device, or an impulse oscillometry device.

In an eleventh aspect combinable with any of the previous aspects, the mouthpiece includes an attachment extension attachable to the PFT device; a user engagement portion; and the bacterial filter coupled between the attachment extension and the user engagement portion.

In a twelfth aspect combinable with any of the previous aspects, the mouthpiece includes a locking mechanism configured to enable attachment of the mouthpiece to the PFT device.

A thirteenth aspect combinable with any of the previous aspects further includes a wireless communication module configured to transmit data that includes at least one of the unique identifier or the patient data between one or more of the mouthpiece, the PFT device, or the control system.

In a fourteenth aspect combinable with any of the previous aspects, the wireless communication module includes an RFID tag associated with the mouthpiece, the RFID tag storing the unique identifier and the patient data; an RFID antenna associated with the mouthpiece to wirelessly transmit signals including at least one of the unique identifier or the patient data from the RFID tag; and an RFID reader associated with the PFT device to identify the mouthpiece based on the signals received from the RFID antenna.

In a fifteenth aspect combinable with any of the previous aspects, the RFID antenna includes a predetermined identification range, and the control system is further operable to perform operations including, based on the mouthpiece and the PFT device being within the predetermined identification range, initiating the interpretation of at least one of the unique identifier or the patient data associated with the mouthpiece.

In a sixteenth aspect combinable with any of the previous aspects, the identification range is between 0 cm and 10 cm, 0 cm and 50 cm, or 0 cm and 1 m.

In a seventeenth aspect combinable with any of the previous aspects, the wireless communication module includes a barcode associated with the mouthpiece, the barcode including the unique identifier and the patient data; and a barcode reader associated with the PFT device to identify the mouthpiece based on the barcode.

In an eighteenth aspect combinable with any of the previous aspects, the wireless communication module includes a Quick Response (QR) code associated with the mouthpiece, the QR code including the unique identifier and the patient data; and a QR reader associated with the PFT device to identify the mouthpiece based on the QR code.

In a nineteenth aspect combinable with any of the previous aspects, the wireless communication module includes at least one of a magnetic, an optical, or an auditory communication module configured to transmit data that includes at least one of the unique identifier or the patient data between one or more of the mouthpiece, the PFT device, or the control system.

In a twentieth aspect combinable with any of the previous aspects, the wireless communication module includes at least one of a magnetic, an optical, or an auditory communication module configured to transmit data that includes at least one of the unique identifier or the patient data between one or more of the mouthpiece, the PFT device, or the control system.

In a twenty-first aspect combinable with any of the previous aspects, the wireless communication module includes a first wireless component attached to the mouthpiece and a second wireless component attached to the PFT device, the control system further operable to perform operations including transmitting the patient data between the first and second wireless components.

In a twenty-second aspect combinable with any of the previous aspects, the control system is further operable to perform operations including retrieving, with the second wireless component, the patient data from a remote database; and sending, to the first wireless component, the retrieved patient data.

In a twenty-third aspect combinable with any of the previous aspects, at least one of the unique identifier or the patient data associated with the mouthpiece is encrypted with a public key.

In a twenty-fourth aspect combinable with any of the previous aspects, the control system is further operable to perform operations including accessing a database communicably coupled with the control system that stores a private key; and decrypting at least one of the unique identifier or the patient data associated with the mouthpiece using the private key.

In a twenty-fifth aspect combinable with any of the previous aspects, the control system includes the database.

In a twenty-sixth aspect combinable with any of the previous aspects, the control system is further operable to perform operations including associating, based on the interpretation of at least one of the unique identifier or the patient data associated with the mouthpiece, the unique identifier with a patient that is uniquely associated with the patient data; and logging the association of the unique identifier with the patient in a database.

In a twenty-seventh aspect combinable with any of the previous aspects, the database is communicably coupled to the control system from a location remote from the PFT system.

In a twenty-eighth aspect combinable with any of the previous aspects, the mouthpiece is a first mouthpiece associated with a first patient, and the control system is further operable to perform operations including receiving a request to perform a pulmonary test with the PFT device on a second patient; determining that the second patient is different than the first patient; and disabling the PFT device based on the determination that the second patient is different than the first patient.

In a twenty-ninth aspect combinable with any of the previous aspects, the control system is further operable to perform operations including interpreting a second unique identifier and second patient data, associated with a second mouthpiece, that is different that the respective unique identifier and patient data; and based on the interpretation, enabling the PFT device to determine a pulmonary function parameter of the second patient.

In a thirtieth aspect combinable with any of the previous aspects, the control system is further operable to perform operations including identifying a testing time range associated with at least one of the mouthpiece or the PFT device; and disabling the PFT device based on the testing time range being greater than a time threshold value.

In a thirty-first aspect combinable with any of the previous aspects, the time threshold value includes a range of at least one of: up to 1 hour, up to 6 hours, or up to 12 hours.

In a thirty-second aspect combinable with any of the previous aspects, the mouthpiece further includes an identification marker that stores permanent data that includes the unique identifier and semi-permanent data that includes the patient data.

In a thirty-third aspect combinable with any of the previous aspects, the unique identifier is associated with at least one of: a serial number, a manufacturer name, a place of manufacture, or a date of manufacture of the mouthpiece.

In a thirty-fourth aspect combinable with any of the previous aspects, the semi-permanent data further includes at least one of a number of mouthpiece uses or a mouthpiece time duration of use.

In a thirty-fifth aspect combinable with any of the previous aspects, the control system further includes or is communicably coupled with a non-volatile read-only memory that stores the permanent data and a non-volatile read-write memory that stores the semi-permanent data.

In other aspects, a computer-implemented method includes providing a detachable part identification module in a medical device, attaching a detachable part, having associated therewith an identification marker, to the medical device at an attachment location, following the attaching, employing the detachable part identification module to identify the identification marker, and based on the identifying, controlling operation of the medical device.

In some implementations, the disclosed subject matter relates to a medical device which has been modified to include a detachable part identification module. The detachable part identification module is configured to detect and identify an identifying marker associated with, or located on, a detachable part fitted into the medical device, and to enable or disable operation of the medical device based on a result of such identification. In some implementations, the medical device is a pulmonary function testing device, and the detachable part is a bacterial/viral filter used in a pulmonary function testing device.

In accordance with an aspect of some implementations, there is provided a method for controlling operation of a medical device, including: providing a detachable part identification module associated with a medical device; attaching a detachable part, having associated therewith an identification marker, to the medical device at an attachment location; following the attaching, employing the detachable part identification module to identify the identification marker; and based on the identifying, controlling operation of the medical device by use of a computerized controller.

In accordance with an aspect of some implementations, there is provided a system for controlling operation of a medical device, including: a detachable part associated with an identification marker, configured to be attached to a medical device at an attachment location; a detachable part identification module associated with the medical device proximate the attachment location and configured to identify the identification marker following attachment of the detachable part at the attachment location; and a controller, functionally associated with the detachable part identification module, configured to control operation of the medical device based on the identification of the identification marker by the detachable part identification module.

In some implementations, the medical device is a hand held medical device. In some implementations, the medical device is a table-top medical device. In some implementations, the medical device is suitable for accommodating a person therein.

In some implementations, the medical device is a pulmonary function testing device, such as a spirometer, a plethysmograph, a diffusion capacity device, a helium dilution device, a nitrogen wash-out device, and an impulse oscillometry device. In some such implementations, the detachable part is a viral/bacterial filter and/or any associated mouthpieces configured to be attached to a pulmonary function testing device.

In some such implementations the detachable part includes an attachment extension configured for attachment to the medical device at the attachment location and a user engagement portion configured to be engaged by the subject being tested. In some such implementations, in which the detachable part is a viral/bacterial filter, the detachable part includes a filtering portion located intermediate the attachment extension and the user engagement portion. In some such implementations, the user engagement portion includes a mouthpiece suitable for the subject to place his mouth over and to blow air into the detachable part towards the filtering portion.

Any suitable material may be used for implementing a detachable part according to the teachings herein. That said, in some implementations, the detachable part is formed of plastic. In some embodiments, the detachable part is recyclable. In some implementations, the detachable part is biodegradable.

In some implementations, the detachable part includes a locking mechanism configured to enable attachment to specific medical devices and to prevent attachment to other medical devices. In some implementations, the locking mechanism can include snap-fit, screws, clamps, clasps, clips, pins, straps, detents, engageable/mating-type surfaces, hook-and-loop, adhesive, and/or any other suitable locking mechanism consistent with this disclosure.

In some implementations, the identification marker includes a passive RFID tag configured to transmit radio frequency signals, and the detachable part identification module includes an RFID antenna configured to receive the radio frequency signals emitted from the RFID tag and an RFID reader configured to identify the RFID tag based on the received signals which can be of any suitable frequency.

In some implementations, the identification marker includes a barcode, and the detachable part identification module includes a barcode reader configured to identify the barcode.

In some implementations, the identification marker includes a Quick Response (QR) code, and the detachable part identification module includes a QR code reader configured to identify the QR code.

In some implementations, the identification marker includes a Bluetooth® transmitter configured to transmit Bluetooth® signals, and the detachable part identification module includes a Bluetooth® receiver configured to receive Bluetooth® signals transmitted by the Bluetooth® transmitter and to identify a source of such signals which can be of any suitable frequency.

In some implementations, the identification marker includes a Wi-Fi transmitter configured to transmit signals, and the detachable part identification module includes a Wi-Fi receiver configured to receive electromagnetic signals transmitted by the Wi-Fi transmitter and to identify a source of such signals which can be of any suitable frequency.

In some implementations, the identification marker includes a magnetic, electronic, optical, and/or auditory identification component suitable to transmit and/or receive appropriate signals (e.g., magnetic fields, electronic signal such as radio, light pulses, sound tones, etc.) and the detachable part identification module includes a transmitter/receiver configured to receive such signals transmitted by the identification component and to identify a source of such signals, which signals can be of any suitable frequency.

In some implementations, the detachable part includes more than one identification marker. In some such implementations, the detachable part includes identification markers of two different types. In some such implementations, a first identification marker is used to identify the detachable part for control of the operation of the medical device, and a second identification marker provides information to an operator of the medical device. Other implementations may use a plurality of identification markers for various purposes consistent with this disclosure.

The identification marker may be of any suitable size to fit the detachable part, as long as it does not interfere with proper operation of the detachable part or of the medical device.

In some implementations, the identification marker is irremovably mounted onto a surface of the detachable part, such as by adhesive, epoxy, soldering, or welding. In some implementations, the identification marker is embedded into the material of the detachable part. In some such implementations, the identification marker is molded into the detachable part.

In some implementations, the identification marker is authenticated by the detachable part identification module. Any suitable software, hardware, or firmware, may be used for carrying out such authentication. For example, open-source/commercially available RFID read/write/programming software may be leveraged to perform functions associated with an identification marker including one or more RFID tags. In a further example, open-source/commercially available optical scanning software can be leveraged where the identification marker is an optical barcode such as a QR code.

In some implementations, the identification marker is encrypted or otherwise protected. In some such implementations, the identification marker is encrypted with a public key, and the detachable part identifying module has access to the private key. In some such embodiments, the private key is stored in the medical device. In some such implementations, the private key is stored remotely to the medical device, and can be accessed via a network.

In some implementations, the detachable part identification module may be configured to identify only the identification marker of a detachable part attached to the medical device at the attachment location. In some such implementations, an identification range of the detachable part identification module is configured to be less than 1 meter. In some such implementations, an identification range of the detachable part identification module is configured to be less than 50 cm. In some such implementations, an identification range of the detachable part identification module is configured to be less than 10 cm.

In some implementations, when the detachable part is attached to the medical device at the attachment location, the detachable part identification module has an uninterrupted line of sight with the identification marker on the detachable part.

In some implementations, the detachable part identification module is functionally associated with a clock, and as part of identifying an identification marker, time-stamps the identification of the identification marker.

In some implementations, the method includes associating an identified detachable part with a specific user. In some such implementations, the detachable part identification module is configured to log the association between an identified detachable part and a specific user, for example in a database.

In some implementations, the database is local to the medical device, and may include information only regarding detachable parts identified by the identification module of the medical device.

In some implementations, the database is a remote database which includes information regarding detachable parts identified in multiple medical devices. For example, the remote database may be a hospital database including information about all detachable parts used in the hospital, a health insurance database including information about all detachable parts used in all facilities of the health insurance provider, a manufacturer database including information about all detachable parts manufactured by the manufacturer, or a regional database including information about all detachable parts used in the region, such as a city or state. In some such embodiments, the association data is transmitted from the medical device to the database via a transmitter, using any suitable wired or wireless communication method. In some such implementations, the association data includes an identification of the medical device used to create the association.

In some implementations, the associating includes associating a user identification number of the specific user with the identified detachable part. In some embodiments, the associating includes associating a user medical record of the specific user with the identified detachable part. In some implementations, the associating includes associating personal user information, such as address, telephone number, date of birth, social security number, and/or passport number of the specific user with the identified detachable part. In some implementations, the associating includes associating a biometric parameter of the specific user, such as the user's fingerprint, palm-print, iris pattern, or facial pattern, with the identified detachable part. In other implementations, any other suitable unique identification indicator capable of uniquely identifying the specific user can be used for this purpose.

In some implementations, controlling operation of the medical device includes disabling operation of the medical device based on detachable part identification results.

In some implementations, the detachable part identification results include association of the identified detachable part with a first subject, when attempting to test a second subject. For example, if the detachable part identification module identifies that a detachable part is associated with a first subject, but the medical professional has accessed medical information or has taken a biometric reading of a second subject, different from the first subject, the medical device is disabled until attachment of a detachable part which is not associated with a subject other than the second subject.

In some implementations, the detachable part identification results include association of the identified detachable part with a testing time prior to a predetermined expiration duration. In some implementations, the expiration duration is no longer than one day. In some implementations, the expiration duration is no longer than 12 hours. In some implementations, the expiration duration is no longer than 6 hours. In some implementations the expiration duration is no longer than one hour. In some implementations, the expiration duration can be of any suitable length.

For example, the detachable parts identification module may identify that a specific detachable part was identified and associated with a specific user at 10:00 am on Jan. 1, 2012. Any attempt to use the same detachable part after passage of the expiration duration, for example, an attempt to use the detachable part at 6:00 pm on Jan. 1, 2012 for the same subject, would result in disabling of the medical device until a new, unexpired, detachable part is attached thereto.

In some implementations, the expiration duration can be used in conjunction with other values to prevent reuse of the same detachable part, mouthpiece, or filter. For example, if an operator uses the same subject information, in the software associated with the medical device, for an entire day and tests several subjects using a single mouthpiece or filter, the time duration may not protect from mouthpiece reuse. In this case, for example, a biometric reading could also be required, thereby preventing mouthpiece reuse even if the time duration had not expired.

In another exemplary scenario, the time duration may expire, but the biometric information indicates that the patient is the same patient, thereby preventing the medical device from being disabled even though the same mouthpiece is being used.

As another example, the time duration and/or biometric reading could be used in conjunction with other values, such as a number of measurements performed with a specific mouthpiece.

In some implementations, the detachable part identification results include lack of identification of the identification marker. For example, if the identification marker is physically damaged or is counterfeit, it would not be identified by the detachable part identification module, leading to disabling of the medical device.

In some implementations, the detachable part identification results include a predetermined color of the identification marker. For example, in some implementations, exposure of the identification marker to bodily fluids of a user, such as saliva, causes the identification marker to change colors. When the detachable part is reattached to the medical device, the detachable part identification module identifies the changed color of the identification marker, leading to disabling of the medical device.

In some implementations, the detachable part identification results include inability to decrypt the identification marker. For example, in some implementations, the identification marker is encrypted using a public key, and the detachable part identification module has access to the private key and attempts to decrypt the identification marker. If the identification marker cannot be decrypted using the private key, the medical device is disabled.

In some implementations, the detachable part identification module compares the identified identification marker to a list of identification markers, for example in a database. In some such implementations, the database is local to the medical device, and includes information regarding detachable parts identified by the identification module of the medical device.

In some such implementations, the database is a remote database which includes information regarding detachable parts identified in multiple medical devices. For example, the remote database may be a hospital database including information about all detachable parts used in the hospital, a health insurance database including information about all detachable parts used in all facilities of the health insurance provider, a manufacturer database including information about all detachable parts manufactured by the manufacturer, or a regional database including information about all detachable parts used in the region, such as a city or state. In some such implementations, the detachable part identification module accesses the database using any suitable wired or wireless communication method.

In some implementations, the detachable part identification module notifies an operator of the medical device that the medical device is disabled. In some such embodiments, the notification indicates the reason for disabling of the medical device. In some such embodiments, the notification indicates how the medical device may be re-enabled. In some such implementations, the notification is a visual notification. In some such implementations, the notification is an audible notification.

In some implementations, the identification marker is physically destroyed following use of the detachable part by a user. For example, in some implementations the medical device includes a destruction mechanism, such as a crushing, tearing, or pinching mechanism, located proximate the attachment location, wherein the destruction mechanism is configured to destroy the identification marker during removal of the detachable part from the attachment location.

In some implementations, the identification marker is disabled or invalidated following use of the detachable part by a user. For example, the detachable part identification module may overwrite the identification marker, reprogram the identification marker, change a validity bit of the identification marker, or erase the identification marker, for example using electrical or mechanical erasing mechanisms.

In some implementations, the controlling is carried out by a processor, such as a processor of a computer, a mobile telephone, a tablet computer, or a PDA. In some embodiments, the processor is integrally formed in the medical device. In some implementations, the processor is external to the medical device.

In some implementations, the processor is configured to run software modules to provide functionality to the medical device. In some such implementations, the software modules are integrally formed in the medical device and are directly associated with the processor. In some such implementations, the software modules are functionally associated with the processor via a network.

In some implementations, the software modules include at least part of the detachable part identification module. In some embodiments, the software modules include a medical test analysis module configured to analyze and provide results of the medical test carried out in the medical device. In some implementations, the software modules include a security module, configured to prevent security and privacy breaches in the medical device, and/or to aid in the identification of an encrypted or otherwise secured identification marker on a detachable part.

In some implementations, the software modules include one or more communication modules configured for communication between the medical device and external devices. In some such implementations, the communication modules include at least one of a printer driver, a wireless networking interface, a wired networking interface, and functionality for communicating with a remote database. Any other suitable communication method is envisioned to be consistent with this disclosure.

In some implementations, the attachment location has a predetermined contour having a specific orientation, and the attachment extension of the detachable part has a matching contour, wherein the detachable part may only be placed at the attachment location in a specific orientation.

In some implementations, the attachment location is part of a detachable part connector element removably fitted in the medical device. The detachable part connector element may be removably fitted into the medical device using any suitable mechanism, including snap-fit, screws, clamps, clasps, clips, pins, straps, detents, engageable/mating-type surfaces, hook-and-loop, adhesive, and/or any other suitable locking mechanism consistent with this disclosure.

In some implementations, the detachable part identification module is included in the detachable part connector element. In some implementations, all modules for control of the identification marker are included in the detachable part connector element. In some such implementations, a destruction mechanism as described herein is included in the detachable part connector element. In some such implementations, invalidation of the identification marker is carried out in the detachable part connector element.

In some implementations, the detachable part connector element includes an authentication mechanism configured, when the detachable part connector element is inserted into the medical device, to interact with an authentication module of the medical device for authentication of the detachable part connector element. In some implementations, following removal of the detachable part connector element from the medical device and reintroduction into the medical device, the authentication module of the medical device re-authenticates the detachable part connector element.

In some implementations, the medical device is connected to other medical devices and/or to a database via a network, for example a wired network or a wireless network. In some such implementations, the other medical devices are in the same facility as the medical device, such as a clinic, medical center, hospital, or hospital network.

In some such implementations, a data collection software module of the medical device is configured to transmit information about the medical device to the other medical devices and/or database. In some such implementations, the information includes information regarding usage of the medical device. In some such embodiments, the information includes information regarding device disabling, such as device disabling statistics and/or metrics. In some such implementations, the information includes information regarding detachable parts, such as an indication of the presence of counterfeit detachable parts, notification of a detachable part recall, or inventory information.

Unless otherwise defined, all technical and scientific terms used herein have the same meaning as commonly understood by one of ordinary skill in the art to which the disclosure pertains. Although methods and materials similar or equivalent to those described herein can be used in the practice or testing of the described subject matter, suitable methods and materials are described below. In case of conflict, the patent specification, including definitions, will control. In addition, the materials, methods, and examples are illustrative only and not intended to be limiting.

Some implementations may involve performing or completing selected tasks manually, automatically, or a combination thereof. Some implementations are implemented with the use of components that include hardware, software, firmware or combinations thereof. In some implementations, some components are general-purpose components such as general-purpose computers or processors. In some implementations, some components are dedicated or custom components such as circuits, integrated circuits or software. For example, some implementations are performed, at least partially, as a plurality of software instructions executed by a data processor, for example which is part of a general-purpose or custom computer. In some implementations, the data processor or computer includes volatile memory for storing instructions and/or data and/or a non-volatile storage, for example, a magnetic hard-disk and/or removable media, for storing instructions and/or data. In some implementations, implementation includes a network (e.g., Internet, Intranet, wired, wireless) connection. In some implementations, implementation includes a user interface, generally including one or more of input devices (e.g., allowing input of commands and/or parameters) and output devices (e.g., allowing reporting parameters of operation and results).

### DESCRIPTION OF DRAWINGS

FIG. 1 is a schematic depiction of an example bacterial/viral filter for use in a pulmonary function testing device;

FIGS. 2A and 2B are schematic depictions of example pulmonary function testing devices;

FIG. 3 is a schematic depiction of an example detachable part identification module of the pulmonary function testing devices of FIGS. 2A and 2B; and

FIGS. 4A and 4B are example schematic flowcharts of a method (or methods) for controlling operation of a pulmonary function testing device.

For simplicity and clarity of illustration, elements shown in the drawings have not necessarily been drawn to scale. For example, the dimensions of some of the elements may be exaggerated relative to other elements for clarity or several physical components may be included in one functional block or element. Moreover, some of the blocks depicted in the drawings may be combined into a single function.

### DETAILED DESCRIPTION

This disclosure generally relates to the field of monitoring use of medical devices, and more particularly, but not exclusively, to methods for monitoring use of a detachable part of a medical device by utilizing identification technologies.

In accordance with an aspect of some implementation, there is provided a method for controlling operation of a medical device, including: providing a detachable part identification module associated with a medical device; attaching a detachable part, having associated therewith an identification marker, to the medical device at an attachment location; following the attaching, employing the detachable part identification module to identify the identification marker; and based on the identifying, controlling operation of the medical device by use of a computerized controller.

In accordance with an aspect of some implementations, there is provided a system for controlling operation of a medical device, including: a detachable part associated with an identification marker, configured to be attached to a medical device at an attachment location; a detachable part identification module associated with the medical device proximate the attachment location and configured to identify the identification marker following attachment of the detachable part at the attachment location; and a controller, functionally associated with the detachable part identification module, configured to control operation of the medical device based on the identification of the identification marker by the detachable part identification module.

In accordance with an aspect of some implementations, there is provided a non-transitory, computer-readable medium, associated with a medical device having associated therewith a detachable part identification module and having attached thereto a detachable part associated with an identification marker, storing computer-readable instructions executable by a computer to: employ the detachable part identification module to identify the identification marker; and based on the identifying, control operation of the medical device.

Although in the illustrated implementations described herein, the medical device is a pulmonary function testing device and the detachable part is a bacterial/viral filter used in a pulmonary function testing device, the methods and systems described herein may be applied to any medical device functionally associated with a detachable part, particularly when a new detachable part must be used for each user or each test.

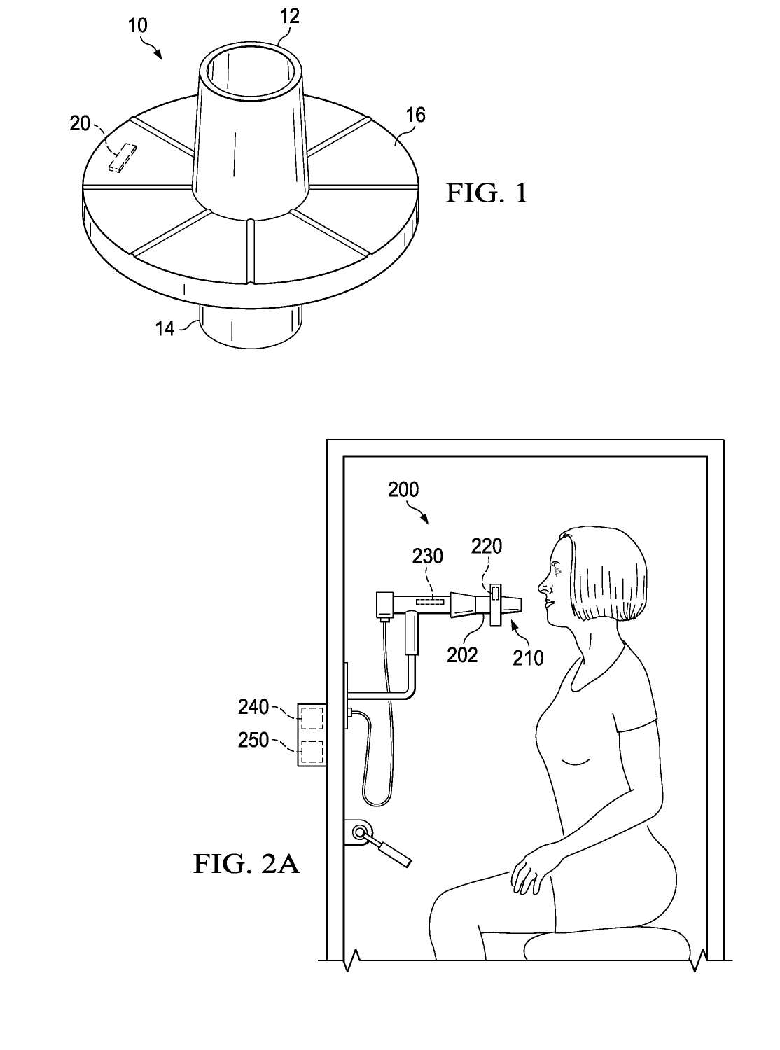

Reference is now made to FIG. 1, which is a schematic depiction of a bacterial/viral filter for use in a pulmonary function testing device according to the teachings herein. As seen in FIG. 1, a bacterial/viral filter 10 includes an attachment extension 14 configured for attachment to a pulmonary function testing device, a user engagement portion 12, and a filtering portion 16 located intermediate attachment extension 14 and user engagement portion 12. User engagement portion 12 includes a mouthpiece suitable for the subject to place his mouth over and to blow air into filter 10 towards a membrane or other suitable filter located in filtering portion 16. Bacterial/viral filter such as filter 10 are commonly used in the art of pulmonary function testing and are commercially available from, for example, Air Safety Ltd. of Morecambe, UK, under catalog number “Spiroguard 2800/21.” Any other suitable bacterial/viral filter may also be used, for example a filter including a rubber mouthpiece fitted onto user engagement portion 12.

Any suitable material may be used for implementing a detachable part according to the teachings herein. That said, in some implementations, the detachable part is formed of plastic. In some implementations, the detachable part is recyclable. In some implementations, the detachable part is biodegradable.

In some implementations, an identification marker 20 is mounted onto filter 10, typically at filtering portion 16. Identification marker 20 is unique to each bacterial/viral filter 10, and is configured to definitively identify filter 10 in order to ensure that filter 10 is suitable for use, as described in detail herein. In some implementations, identification marker 20 also contains information that is unique to a patient such as patient information, doctor information, measurement data, and filter use information, as described in detail herein.

In some implementations, identification marker 20 is irremovably mounted onto an external surface of filtering portion 16, such as by adhesive, epoxy, soldering, or welding. In some implementations, identification marker 20 is embedded into the material forming filtering portion 16, for example by being molded into the material. Identification marker 20 may be of any suitable size consistent with this disclosure.

In some implementations, identification marker 20 includes a transmitter for transmitting a communication signal to a portion of a medical device as described herein. For example, identification marker 20 may be a passive RFID tag, a Bluetooth® transmitter, or a Wi-Fi transmitter.

In some implementations, identification marker 20 includes a marker which is readable by a reading device. For example, identification marker 20 may be a barcode or a Quick Response (QR) code.

In some implementations, identification marker 20 includes a receiver for receiving a communication signal from a medical device as described herein.

In some implementations, identification marker 20 includes read-only non-volatile memory for storage of information to uniquely identify filter 10.

In some implementations, identification marker 20 includes read-write non-volatile memory for storage of additional information and for updating information on identification marker 20 based on information received from the identification marker receiver.

In some implementations, in addition to uniquely identifying filter 10, identification marker 20 may include information regarding an expiration time of filter 10, a user to whom filter 10 was assigned, manufacturing information for filter 10, and any other information that may be needed by a medical professional using filter 10 and/or by a validation mechanism validating filter 10. The additional information may also include filter use information (e.g., duration of use, timestamps, number of uses, etc.), patient information (e.g., name, birthdate, social security number, etc.), doctor information (e.g., name, etc.), insurer information (e.g., provider name, policy number, etc.), and measurement data (e.g., spirometry, lung volumes, etc.).

In some implementations, identification marker 20 may only be used to uniquely identify filter 10, and a second identification marker (not shown) includes additional information useful for validation of filter 10 or for medical use of filter 10. The second identification marker may be of the same type as identification marker 20, or may be of a different type. In some implementations, the single identification marker 20 contains both information to uniquely identify the filter and additional information useful for validation of filter 10 or for medical use of filter 10. In some implementations, identification marker 20 is partitioned into non-volatile memory that stores permanent, read-only information and non-volatile memory that stores updatable, read-write information.

In some implementations, the permanent, read-only information (e.g., including serial number, manufacturer name, and place and date of manufacturing) is written to the non-volatile memory at the factory before shipment of the product. In some implementations, the updatable, read-write information is initiated at the factory, such as setting the number of uses to zero and the time duration of use to zero. In some implementations, the updatable information is updated in real-time on identification marker 20 before, during, or after use of filter 10 within a pulmonary function laboratory.

In some implementations, identification marker 20 is authenticated prior to use, as described in further detail herein. In some implementations, the information included in identification marker 20 is encrypted or otherwise protected. Any suitable encryption or protection method may be used, such as public key encryption.

In some implementations, bacterial/viral filter 10 includes a locking mechanism (not shown) configured to enable attachment only to one or more medical devices of a specific type and to prevent attachment to other medical devices, for example other makes/third-party medical devices.

The locking mechanism, in some implementations, can include snap-fit, screws, clamps, clasps, clips, pins, straps, detents, engageable/mating-type surfaces, hook-and-loop, adhesive, and/or any other suitable locking mechanism consistent with this disclosure. In some implementations, the act of attaching the filter 10 to a medical device results in a physical change, temporary or permanent, in the filter 10 that can prevent, once filter 10 is removed from the medical device, re-attachment of the filter to the same medical device and/or to a different medical device. For example, attachment of filter 10 to the medical device may break a protrusion (not shown) off filter 10, which protrusion is necessary to unlock a limiting element (not shown) allowing initial insertion of the filter 10 into the medical device. Other possible changes include tearing, crushing, punching one or more holes, deforming, and the like.

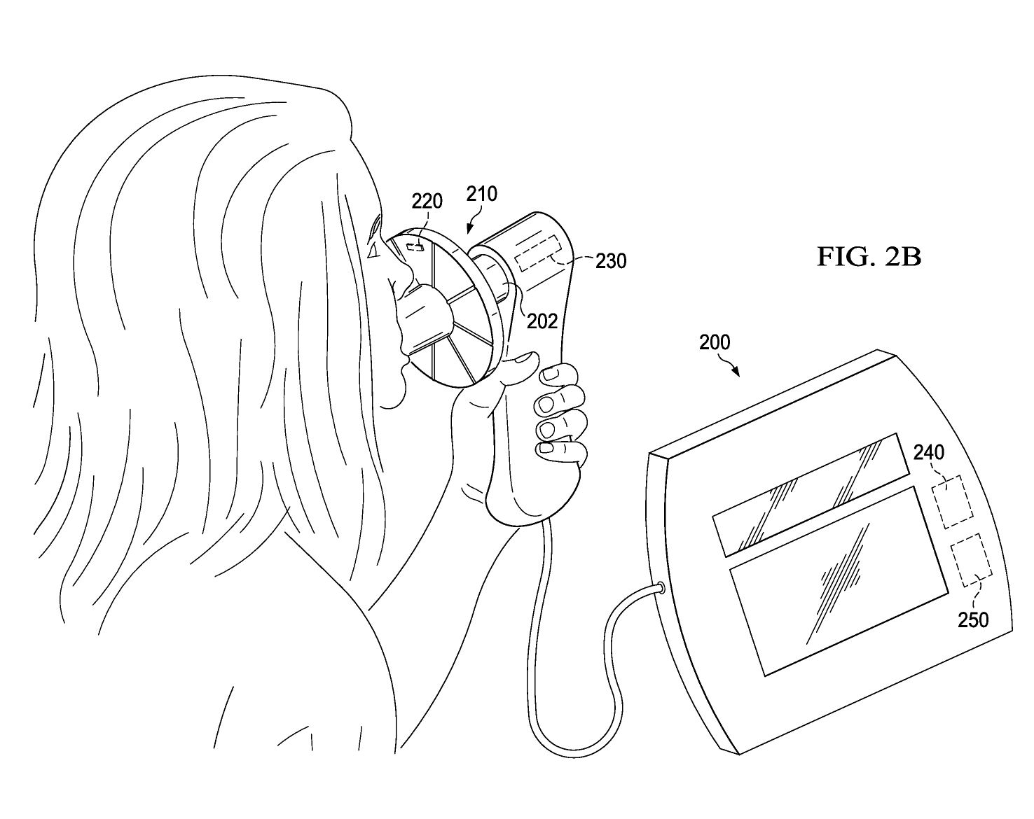

Reference is now made to FIGS. 2A and 2B, which are schematic depictions of pulmonary function testing devices according to the teachings herein. As seen in FIGS. 2A and 2B, a pulmonary testing device 200 has connected thereto at an attachment location 202 a bacterial/viral filter 210, similar to filter 10 described herein with reference to FIG. 1. As seen clearly in FIG. 2B, filter 210 includes an identification marker 220 substantially as described herein with reference to FIG. 1.

In FIG. 2A pulmonary testing device 200 is a whole body plethysmograph, known in the art as a “body box”, and in FIG. 2B pulmonary testing device 200 is a portable spirometer. Thus, the features described herein may be applied to any suitable medical device, including portable medical devices and non-portable medical devices, in different levels of complexity. In some aspects, the features described herein may be applied to the devices described in U.S. patent application Ser. Nos. 12/670,661, 12/830,955, 13/146,295, and 13/808,868, which are incorporated by reference herein.

Pulmonary function testing device 200 includes a detachable part identification module 230, configured to read or interact with identification marker 220 in order to identify the validity of filter 210. Detachable part identification module 230 typically is located proximate filter 210 and/or proximate identification marker 220, in order to facilitate proper function of detachable part identification module 230.

In some implementations, in which identification marker 220 includes a passive RFID tag configured to transmit signals, detachable part identification module 230 includes an RFID antenna configured to receive signals of any suitable frequency emitted from the RFID tag and an RFID reader configured to identify the RFID tag. In some such implementations, the frequency of the RFID signals is set within a suitable range such that the antenna may only receive signals from identification marker 220 located on a filter 210 currently connected to device 200 and not from identification markers of other filters with the same RFID signal range or with a different RFID signal range, for example other filters stored in a room where pulmonary function testing, using device 200 and/or other medical devices and/or medical device types, takes place. In some implementations, each medical device can be set, calibrated, programmed, or otherwise suitably configured to accept one or more specific RFID signal types and/or frequencies.

In some implementations, in which identification marker 220 includes a barcode, detachable part identification module 230 includes a barcode reader configured to identify the barcode. In some implementations, in which identification marker 220 includes a Quick Response (QR) code, detachable part identification module 230 includes a QR code reader configured to identify the QR code. In some such implementations, detachable part identification module 230 includes an image capturing mechanism for capturing an image of the barcode or QR code, and the image capturing mechanism has an uninterrupted line of sight to the barcode or QR code.

In some implementations, in which identification marker 220 includes a Bluetooth® transmitter configured to transmit signals, detachable part identification module 230 includes a Bluetooth® receiver configured to receive Bluetooth® signals transmitted by the Bluetooth® transmitter and to identify a source of such signals. In some such implementations, the frequency of such signals is set to be within a suitable range such that the Bluetooth® receiver may only receive signals from identification marker 220 located on a filter 210 currently connected to device 200 and not from identification markers of other filters with the same Bluetooth® signal range or with a different Bluetooth® signal range, for example other filters stored in a room where pulmonary function testing, using device 200 and/or other medical devices and/or medical device types, takes place. In some implementations each medical device can be set, calibrated, programmed, or otherwise suitably configured to accept one or more specific Bluetooth® signal types and/or frequencies.

In some implementations, in which identification marker 220 includes a Wi-Fi transmitter configured to transmit signals, detachable part identification module 230 includes a Wi-Fi receiver configured to receive Wi-Fi signals transmitted by the Wi-Fi transmitter and to identify a source of such signals. In some such implementations, the frequency of such signals is set to be within a suitable range such that the Wi-Fi receiver may only receive signals from identification marker 220 located on a filter 210 currently connected to device 200 and not from identification markers of other filters with the same Wi-Fi range or with a different Wi-Fi range, for example other filters stored in a room where pulmonary function testing, using device 200 and/or other medical devices and/or medical device types, takes place. In some implementations each medical device can be set, calibrated, programmed, or otherwise suitably configured to accept one or more specific Wi-Fi signal types and/or frequencies.

In some implementations, in which identification marker 220 includes a receiver configured to receive signals, detachable part identification module 230 includes a transmitter configured to transmit signals to the identification marker receiver. As above, the technology used may be RFID, Bluetooth, Wi-Fi, or any suitable technologies for transmitting and receiving signals.

In some implementations, the identifying information within the identification marker 220 is stored on non-volatile memory. In some implementations, the non-volatile memory includes one or many microchips within an RFID tag, which may be set to read-only, read-write, or write-once read-many. In some implementations, the non-volatile memory includes EEPROM, or any suitable memory as described herein.

In some aspects, detachable part identification module 230 may be configured to identify only the identification marker 220 of a detachable part 210 attached to the medical device 220 at attachment location 202. In some implementations, an identification range of detachable part identification module 230 is configured to be less than 1 meter. In some implementations, an identification range of detachable part identification module 230 is configured to be less than 50 cm. In some implementations, an identification range of detachable part identification module 230 is configured to be less than 10 cm.

In some aspects, detachable part identification module 230 may be configured to only identify the single detachable part 210 that is attached to the medical device 220 at attachment location 202, and to not identify other detachable parts stored in the same room. In some implementations, detection of the presence of a detachable part attached to the medical device is required before the detachable part identification module is initiated to read the detachable part. In some implementations, attachment of a detachable part 210 to the medical device 220 at attachment location 202 results in a mechanical or electrical deformation that serves as the initial detection of the detachable part by the medical device. In some implementations, detachable part identification module 230 may read identification marker 220 only after the initial detection is received. In some implementations, detachable part identification module 230 may be initiated to read identification marker 220 only after the user provides an input to a graphical user interface. In some implementations, the duration of detection of a detachable part by the identification module is configured to be less than 10 seconds. In some implementations, the duration of detection of a detachable part by the identification module is configured to be less than 5 seconds. In some implementations, the duration of detection of a detachable part by the identification module is configured to be less than 1 second.

Detachable part identification module 230 is functionally associated with a controller 240 of medical device 200, which controller can disable medical device 200 if detachable part identification module 230 indicates that the currently attached filter 210 is invalid or unidentified, as will be described in further detail herein.

In some implementations, detachable part identification module 230 is functionally associated with a clock 250, and is configured to timestamp a time of connection of filter 210 to device 200 based on a time indicated by clock 250.

In some implementations, medical device 200 includes a destruction mechanism (not shown) for physically destroying identification marker 220 following use of detachable part 210 by a user. The destruction mechanism may be any suitable destruction mechanism, such as a crushing, tearing, or pinching mechanism, located proximate attachment location 202.

In some implementations, medical device 200 includes an invalidation mechanism (not shown) for invalidating identification marker 220 following use of detachable part 210 by a user. The invalidation mechanism may be any suitable mechanism, such as a module for overwriting the identification marker, reprogramming the identification marker, re-encrypting the identification marker with a random key, changing a validity bit of the identification marker, or erasing the identification marker, for example using electrical or magnetic signals. In some implementations, the invalidation mechanism forms part of detachable part identification module 230. In some implementations, the invalidation mechanism includes erasing all confidential information from the identification marker, including patient information, measurement data, and doctor information.

In some implementations, the invalidation mechanism is not associated with the medical device but is instead a separate device specifically designed to invalidate an identification marker 220 of detachable part 210. In some implementations, the invalidation mechanism is housed within an invalidation wand (not shown) that can be waved over an identification marker to invalidate the marker. In some implementations, the invalidation mechanism occurs after the detachable part is removed from the medical device.

In some implementations, attachment location 202 has a predetermined contour having a specific orientation, and the attachment extension of the detachable part has a matching contour, wherein the detachable part may only be placed at the attachment location in a specific orientation.

In some implementations, attachment location 202 is part of a detachable part connector element (not shown) removably fitted into medical device 200. The detachable part connector element may be removably fitted into the medical device using any suitable mechanism, including snap-fit, screws, clamps, clasps, clips, pins, straps, detents, engageable/mating-type surfaces, hook-and-loop, adhesive, and/or any other suitable fitting mechanism consistent with this disclosure.

In some implementations, detachable part identification module 230 forms part of the detachable part connector element. In some implementations, the detachable part connector element includes an authentication mechanism configured, when the detachable part connector element is inserted into medical device 200, to interact with an authentication module of medical device 200 for authentication of the detachable part connector element. In some embodiments, following removal of the detachable part connector element from medical device 200 and reintroduction into the medical device, the authentication module of medical device 200 re-authenticates the detachable part connector element.

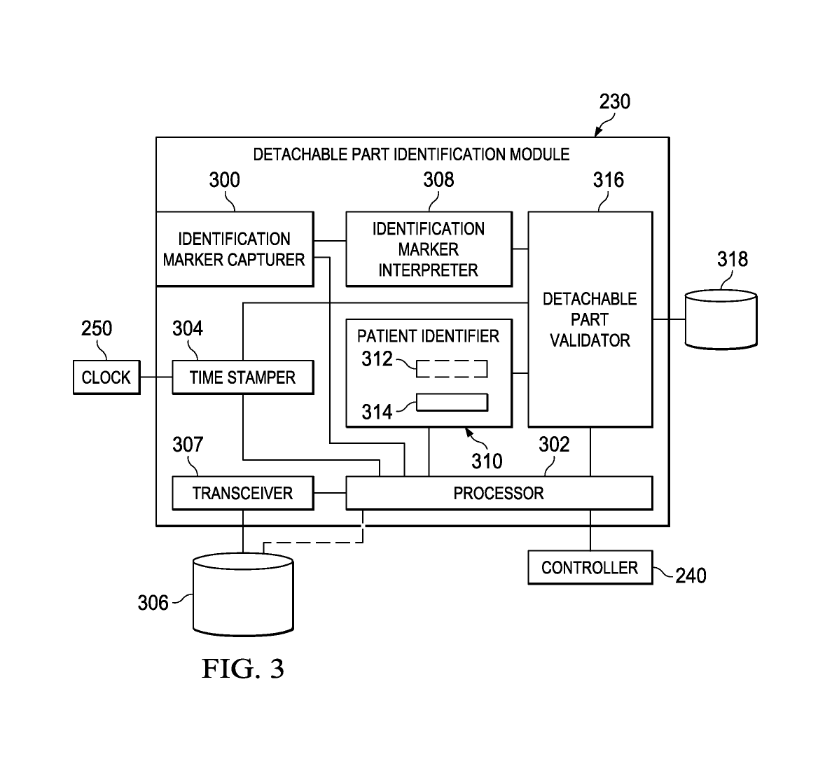

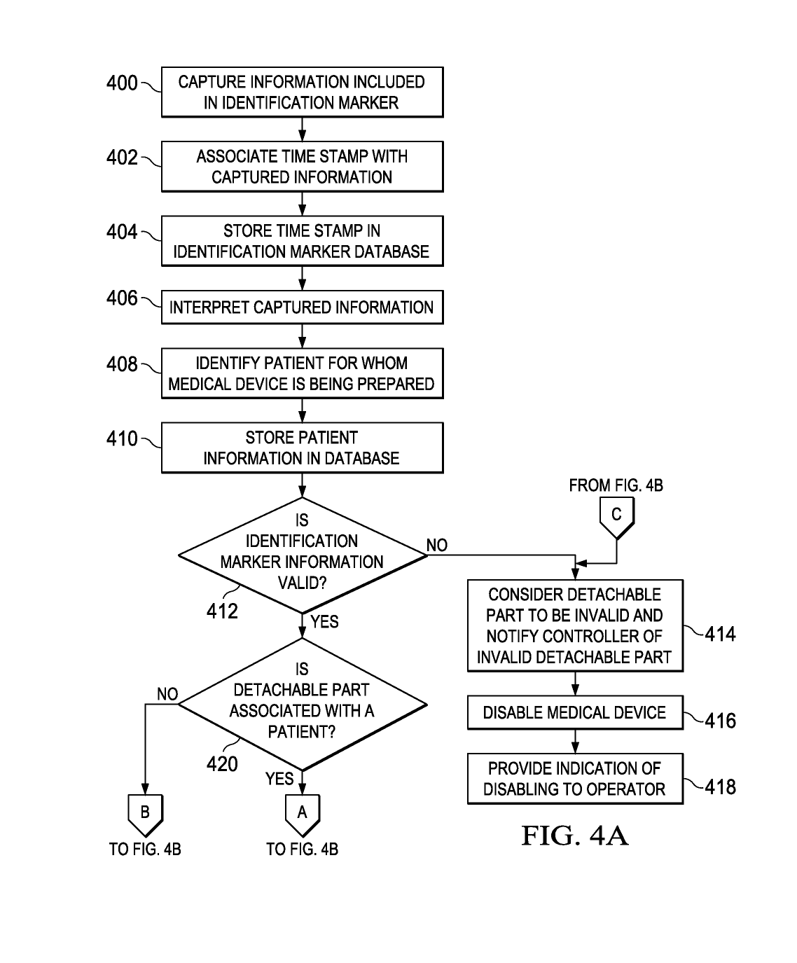

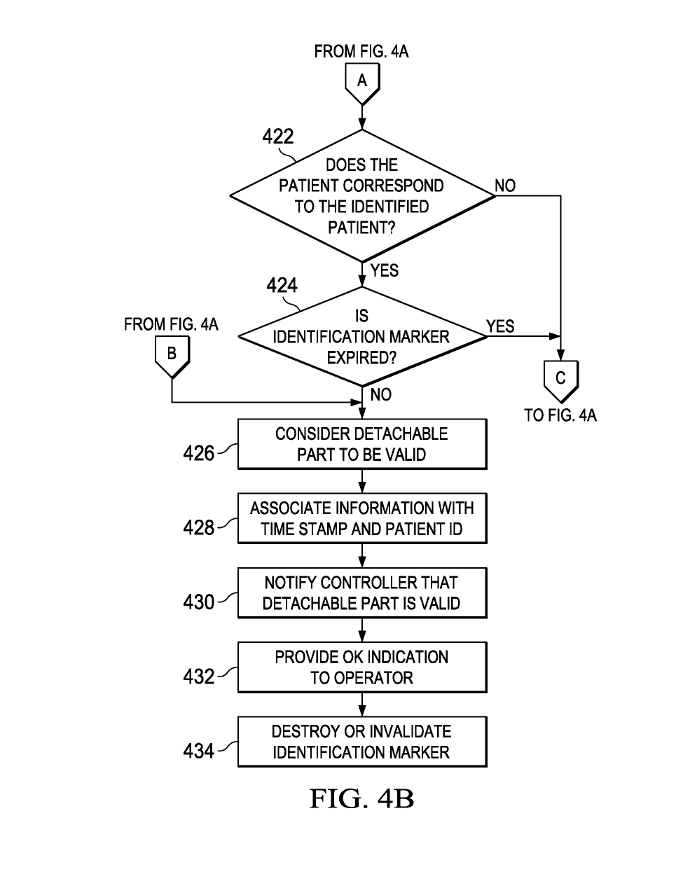

Reference is now made to FIG. 3, which is a schematic depiction of detachable part identification module 230 of pulmonary function testing devices 200 of FIGS. 2A and 2B, according to the teachings herein, and to FIGS. 4A and 4B, which, when taken together, are a schematic flowchart of a method for controlling operation of a pulmonary function testing device according to the teachings herein.

As seen in FIG. 3, detachable part identification module 230 includes an identification marker capturer 300, configured to capture the information included in identification marker 220 upon attachment of filter 210 at attachment location 202, as seen at step 400 in FIG. 4A. Typically, identification marker capturer 300 is a hardware component, such as a barcode reader, a QR code reader, or a communication signal receiver such as an RFID antenna, a Bluetooth® receiver, or a Wi-Fi receiver.

As seen at step 402 in FIG. 4A, upon capturing of identification marker information by identification marker capturer 300, a processor 302 associated with or forming part of detachable mark identification module 230 activates a time-stamper 304 to associate a time stamp, determined using clock 250, with the captured identification marker information. In some implementations, processor 302 stores the time stamp in an identification marker database 306, implemented, for example, as a SQL relational database, as seen at step 404 of FIG. 4A.

In some implementations, identification marker database 306 is local to medical device 200, and contains information about identification markers used in device 200. In other implementations, identification marker database 306 is remote from medical device 200, and processor 302 stores the time stamp in database 306 by transmitting the time stamp to database 306 using a transceiver 307. Transceiver 307 may use any suitable form of communication including wired communication and wireless communication. In some such implementations, transceiver 307 additionally transmits to database 306 an identification of the specific medical device 200 at which the time stamp was obtained.

In some implementations, remote database 306 includes information regarding detachable parts identified in multiple medical devices. For example, database 306 may be a hospital database including information about all detachable parts used in the hospital, a health insurance database including information about all detachable parts used in all facilities of the health insurance provider, a manufacturer database including information about all detachable parts manufactured by the manufacturer, or a regional database including information about all detachable parts used in the region, such as a city or state.

In some implementations, information captured by identification marker capturer 300 is transferred to an identification marker interpreter 308, for interpretation of the captured information, as seen at step 406 of FIG. 4A. In some implementations, the identification marker interpreter is a software component, such as a RFID reader which interprets information included in RFID signals, or a Bluetooth® or Wi-Fi signal interpreter configured to identify the origin of Bluetooth® or Wi-Fi signals.

In some implementations, identification marker interpreter 308 is configured to decrypt information included in identification marker 220. In some such embodiments, identification marker 220 is encrypted with a public key, and identification marker interpreter 308 has access to the corresponding private key. In some embodiments, the private key is stored in medical device 200, for example in a security software module. In some implementations, the private key is stored remotely to medical device 200, and can be accessed via a network, for example using transceiver 307.

In some implementations, upon interpretation of the identification marker information, processor 302 stores the interpreted information in database 306 in association with the stored time stamp.

At step 408 of FIG. 4A, processor 302 activates a patient identifier 310, configured to identify a patient for whom medical device 200 and filter 210 are being prepared. In some implementations, processor 302 stores the patient information in database 306, for example as part of a relational database table, as seen at step 410 of FIG. 4A. It is appreciated that patient identification may occur prior to, concurrently with, or after capturing and/or interpretation of the identification marker information.

In some implementations, patient identifier 310 includes a hardware component 312, configured to scan a biometric identifier of the patient, such as a fingerprint, a palm print, or an iris pattern, and a software component 314 configured to obtain a patient identification of a patient to whom the biometric identifier belongs. For example, software component 314 may access a biometric identifier database (not shown) to obtain patient information such as the patients name, address, date of birth, patient number, social security number, passport number, or other information suitable for uniquely identifying the patient.

In other implementations, patient identifier may only include software component 314, for example implemented as a separate process or thread run on processor 302, which accesses software elements associated with medical device 200 to identify the patient. For example, software component 314 may access a user interface in which a medical professional provides patient identification information.

Once the information of the identification marker and of the patient are obtained, processor 302 runs a detachable part validator 316, for example implemented as a separate process or thread, to determine whether the detachable part is valid for use at this time with this patient.

At step 412 of FIG. 4A, detachable part validator 316 authenticates the information of the identification marker to determine its validity. An identification marker may be considered invalid in many instances. For example, if detachable part validator 316 does not recognize the information included in the marker, as may occur if counterfeit identification markers are used, detachable part 210 is considered invalid. As another example, if no identification marker indication is captured by capturer 300 detachable part 210 is considered invalid. As a further example, if identification marker interpreter 308 was not able to decrypt the information included in the marker, or if identification marker interpreter 308 found a mistake in a validity bit of the marker, detachable part 210 is considered invalid.

In some implementations, detachable part validator 316 accesses a database 318, which may be part of medical device 200 or may be external to medical device 200, in order to ascertain the validity of detachable part 210. In some such implementations, for example in implementations in which the identification marker is an RFID tag, database 318 may list all valid RFID tag numbers, such that if detachable part validator 316 does not locate the identified number in database 318, detachable part 210 is considered invalid.

In some implementations, information on the validity of the detachable part may be stored on the detachable part itself. The detachable part identification marker may have a re-writable bit that describes whether or not the detachable part is valid. For example, upon use of a single-use detachable part, the medical device identification module may re-write the validity bit to change its status to invalid at the end of the measurement procedure. Upon re-using the same detachable part, the medical device identification module reads the validity bit and determines that the detachable part is invalid. In this embodiment, it is not required to compare the detachable part identification marker to a database since all required information is stored on the detachable part itself. In some implementations, the rules governing the status of the validity bit may be based on the number of uses of the detachable part, the time duration of use, or a combination of the two. In some implementations, the validity bit is updated and re-written after each measurement is performed.

In some implementations, neither a validity bit nor a database exists. Instead, the validity of the detachable part is determined by the detachable part identification module 230 based on reading all information stored locally on the detachable part. Validity is then determined by detachable part validator 316 based on a rule set using the detachable part information. In some implementations, the rule set may include the number of uses, time duration of use, time stamp, and patient information.

As seen at step 414 of FIG. 4B, when detachable part 210 is invalid, processor 302 notifies controller 240 that the detachable part is invalid, and controller 240 disables use of medical device 200 at step 416 of FIG. 4B. In some implementations, controller 240 indicates to an operator of medical device 200 that the device is disabled at step 418 of FIG. 4B. Any suitable indication may be used, for example a visual indication presented on a display of medical device 200, or an audible indication. In some such implementations, the indication informs the operator why the medical device has been disabled.

If the information obtained from identification marker 220 is determined to be valid, detachable part validator 316 checks whether or not detachable part 210 is associated with a patient, at step 420 of FIG. 4B. In some implementations, association of a specific detachable part 210 having a unique identification marker 220 with a patient is logged in identification marker database 306, and detachable part validator 316 determines the existence of such association between a patient and the detachable part by accessing database 306, locally or remotely, as described herein.

In some implementations, patient information is stored on the detachable part itself and is compared to information entered by a medical professional into a user interface or information retrieved on a database connectable to the medical device. For example, software component 314 may access a user interface in which a medical professional provides patient identification information. The detachable part validator 316 confirms whether or not the patient information read from the detachable part is associated with the patient information entered or read on the medical device. In some implementations, biometric information, such as a fingerprint scan, is stored on the detachable part. The detachable part validator 316 confirms whether the stored biometric information matches a new scan of a biometric identifier that is performed when the detachable part is attached to the medical device.

As seen at step 422 of FIG. 4B, if identification marker 220 is associated with a patient, detachable part validator 316 determines whether the patient with whom the identification marker 220 is associated corresponds to the patient identified by patient identifier 310. If identification marker 220 is associated with a patient that does not correspond to the patient identified by patient identifier 310, detachable part 210 carrying identification marker 220 is considered invalid, as seen at step 414 described herein, and the medical device 200 is disabled as seen at step 416 and as described herein.

Otherwise, if identification marker 220 is associated with the patient identified by patient identifier 310, detachable part validator 316 determines whether or not the identification marker has expired, at step 424 of FIG. 4B. In order to determine whether or not the identification marker has expired, detachable part validator 316 computes the duration that passed since the identification marker 220 was associated with the patient, and compares the computed duration to a predetermined expiration duration. If the computed duration is greater than the predetermined expiration duration, the detachable part 210 carrying identification marker 220 is considered invalid, as seen at step 414 described herein, and the medical device 200 is disabled as seen at step 416 and as described herein. In some implementations, the expiration duration is no longer than 12 hours. In some implementations, the expiration duration is no longer than 6 hours. In some implementations the expiration duration is no longer than one hour. In some implementations, the expiration duration can be of any suitable length.

In some implementations, detachable part validator 316 checks whether the identification marker is in a color indicating that it had been used. For example, in some implementations, exposure of the identification marker to bodily fluids of a user, such as saliva, causes the identification marker to change from a first color indicating a new marker, to a second color indicating a used marker, similarly to litmus paper. When detachable part validator 316 identifies the second color, the detachable part 210 carrying identification marker 220 is considered invalid, as seen at step 414 described herein, and the medical device 200 is disabled as seen at step 416 and as described herein.

If at step 420 detachable part validator 316 determines that the detachable part is not associated with any patient, or at step 424 detachable part validator 316 determines that the detachable part is associated with the correct patient and is not expired, the detachable part 210 is considered valid, as seen at step 426 of FIG. 4B.

Following validation of detachable part 210, processor 302 associates the information captured by identification mark capturer 300 to an identification marker interpreter 308 with the time stamp obtained from time stamper 304 and with the patient information obtained from patient identifier 310, as seen at step 428 of FIG. 4B. At step 430 of FIG. 4B, processor 302 notifies controller 240 that the detachable part is valid, and controller 240 indicates to an operator of medical device 200 that testing may begin, as seen at step 432 of FIG. 4B.

In some implementations, following use of detachable part 210, either once, or multiple times for the same patient, identification marker 220 is destroyed or disabled, as described herein with reference to FIGS. 2A and 2B.

In some implementations, detachable part 210 is destroyed or disabled after a fixed duration of time following the initial use of the detachable part. In some implementations, detachable part 210 is destroyed or disabled after a fixed number of uses recorded by the medical device. In some implementations, the detachable part has already been removed from the medical device without already having been destroyed or disabled.

In some implementations, the detachable part 210 is initiated prior to attachment to the medical device. That is, a separate device such as an RFID reader and transmitter attached to a suitable computer is used to load information, such as patient information, onto an un-used detachable part. In some implementations, the patient information stored on the detachable part is read upon attachment to a first medical device. In some implementations, the patient information stored on the detachable part after use on a first medical device is read when detachable part is attached to a second medical device. In some implementations, the patient information read by the medical device is used to associate the measurement data with the proper patient within a database or computer system. In some implementations, detachable part 210 remains valid for multiple measurements and for use on multiple medical devices.

Certain features of the described subject matter, which are, for clarity, described in the context of separate implementations, may also be provided in combination in a single implementations. Conversely, various features of the disclosed subject matter, which are, for brevity, described in the context of a single implementations, may also be provided separately or in any suitable subcombination or as suitable in any other described implementations. Certain features described in the context of various implementations are not to be considered essential features of those implementations, unless the implementation is inoperative without those elements.

Although the disclosed subject matter has been described in conjunction with specific implementations thereof, it is evident that many alternatives, modifications and variations will be apparent to those skilled in the art. Accordingly, it is intended to embrace all such alternatives, modifications and variations that fall within the scope of the appended claims.

Implementations of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Implementations of the subject matter described in this specification can be implemented as one or more computer programs, e.g., one or more modules of computer program instructions encoded on a tangible, non-transitory computer-storage medium for execution by, or to control the operation of, data processing apparatus. Alternatively or in addition, the program instructions can be encoded on an artificially-generated propagated signal, e.g., a machine-generated electrical, optical, or electromagnetic signal that is generated to encode information for transmission to suitable receiver apparatus for execution by a data processing apparatus. The computer-storage medium can be a machine-readable storage device, a machine-readable storage substrate, a random or serial access memory device, or a combination of one or more of them.

The term “data processing apparatus” refers to data processing hardware and encompasses all kinds of apparatus, devices, and machines for processing data, including by way of example a programmable processor, a computer, or multiple processors or computers. The apparatus can also be or further include special purpose logic circuitry, e.g., a central processing unit (CPU), a FPGA (field programmable gate array), or an ASIC (application-specific integrated circuit). In some implementations, the data processing apparatus and/or special purpose logic circuitry may be hardware-based and/or software-based. The apparatus can optionally include code that creates an execution environment for computer programs, e.g., code that constitutes processor firmware, a protocol stack, a database management system, an operating system, or a combination of one or more of them. The present disclosure contemplates the use of data processing apparatuses with or without conventional operating systems, for example LINUX, UNIX, WINDOWS, MAC OS, ANDROID, IOS or any other suitable conventional operating system.

A computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a computing environment. A computer program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub-programs, or portions of code. A computer program can be deployed to be executed on one computer or on multiple computers that are located at one site or distributed across multiple sites and interconnected by a communication network. While portions of the programs illustrated in the various figures are shown as individual modules that implement the various features and functionality through various objects, methods, or other processes, the programs may instead include a number of sub-modules, third party services, components, libraries, and such, as appropriate. Conversely, the features and functionality of various components can be combined into single components as appropriate.

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., a CPU, a FPGA, or an ASIC.

Computers suitable for the execution of a computer program include, by way of example, can be based on general or special purpose microprocessors or both, or any other kind of CPU. Generally, a CPU will receive instructions and data from a read-only memory (ROM) or a random access memory (RAM) or both. The essential elements of a computer are a CPU for performing or executing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a global positioning system (GPS) receiver, or a portable storage device, e.g., a universal serial bus (USB) flash drive, to name just a few.

Computer-readable media (transitory or non-transitory, as appropriate) suitable for storing computer program instructions and data include all forms of non-volatile memory, media and memory devices, including by way of example semiconductor memory devices, e.g., erasable programmable read-only memory (EPROM), electrically-erasable programmable read-only memory (EEPROM), and flash memory devices; magnetic disks, e.g., internal hard disks or removable disks; magneto-optical disks; and CD-ROM, DVD+/−R, DVD-RAM, and DVD-ROM disks. The memory may store various objects or data, including caches, classes, frameworks, applications, backup data, jobs, web pages, web page templates, database tables, repositories storing business and/or dynamic information, and any other appropriate information including any parameters, variables, algorithms, instructions, rules, constraints, or references thereto. Additionally, the memory may include any other appropriate data, such as logs, policies, security or access data, reporting files, as well as others. The processor and the memory can be supplemented by, or incorporated in, special purpose logic circuitry.

To provide for interaction with a user, implementations of the subject matter described in this specification can be implemented on a computer having a display device, e.g., a CRT (cathode ray tube), LCD (liquid crystal display), or plasma monitor, for displaying information to the user and a keyboard and a pointing device, e.g., a mouse, trackball, or trackpad by which the user can provide input to the computer. Input may also be provided to the computer using a touchscreen, such as a tablet computer surface with pressure sensitivity, a multi-touch screen using capacitive or electric sensing, or other type of touchscreen. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

The term “graphical user interface,” or GUI, may be used in the singular or the plural to describe one or more graphical user interfaces and each of the displays of a particular graphical user interface. Therefore, a GUI may represent any graphical user interface, including but not limited to, a web browser, a touch screen, or a command line interface (CLI) that processes information and efficiently presents the information results to the user. In general, a GUI may include a plurality of user interface (UI) elements, some or all associated with a web browser, such as interactive fields, pull-down lists, and buttons operable by the business suite user. These and other UI elements may be related to or represent the functions of the web browser.

Implementations of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described in this specification, or any combination of one or more such back-end, middleware, or front-end components. The components of the system can be interconnected by any form or medium of wireline and/or wireless digital data communication, e.g., a communication network. Examples of communication networks include a local area network (LAN), a radio access network (RAN), a metropolitan area network (MAN), a wide area network (WAN), Worldwide Interoperability for Microwave Access (WIMAX), a wireless local area network (WLAN) using, for example, 802.11 a/b/g/n and/or 802.20, all or a portion of the Internet, and/or any other communication system or systems at one or more locations. The network may communicate with, for example, Internet Protocol (IP) packets, Frame Relay frames, Asynchronous Transfer Mode (ATM) cells, voice, video, data, and/or other suitable information between network addresses.

The computing system can include clients and servers. A client and server are generally remote from each other and typically interact through a communication network. The relationship of client and server arises by virtue of computer programs running on the respective computers and having a client-server relationship to each other.

In some implementations, any or all of the components of the computing system, both hardware and/or software, may interface with each other and/or the interface using an application programming interface (API) and/or a service layer. The API may include specifications for routines, data structures, and object classes. The API may be either computer language independent or dependent and refer to a complete interface, a single function, or even a set of APIs. The service layer provides software services to the computing system. The functionality of the various components of the computing system may be accessible for all service consumers via this service layer. Software services provide reusable, defined business functionalities through a defined interface. For example, the interface may be software written in JAVA, C++, or other suitable language providing data in extensible markup language (XML) format or other suitable format. The API and/or service layer may be an integral and/or a stand-alone component in relation to other components of the computing system. Moreover, any or all parts of the service layer may be implemented as child or sub-modules of another software module, enterprise application, or hardware module without departing from the scope of this disclosure.

While this specification contains many specific implementation details, these should not be construed as limitations on the scope of any invention or on the scope of what may be claimed, but rather as descriptions of features that may be specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation and/or integration of various system modules and components in the implementations described above should not be understood as requiring such separation and/or integration in all implementations, and it should be understood that the described program components and systems can generally be integrated together in a single software product or packaged into multiple software products.

Particular implementations of the subject matter have been described. Other implementations, alterations, and permutations of the described implementations are within the scope of the following claims as will be apparent to those skilled in the art. For example, the actions recited in the claims can be performed in a different order and still achieve desirable results.

Accordingly, the above description of example implementations does not define or constrain this disclosure. Other changes, substitutions, and alterations are also possible without departing from the spirit and scope of this disclosure.

1. A pulmonary function testing (PFT) system, including:
a PFT device operable to receive a respiratory airflow from a patient through an airflow chamber;
a mouthpiece coupled to the PFT device and in airflow communication with the airflow chamber, the mouthpiece comprising a bacterial filter and associated with a unique identifier and patient data; and
a control system communicably coupled with the mouthpiece and the PFT device, the control system configured to perform operations comprising:
interpreting at least one of the unique identifier or the patient data associated with the mouthpiece; and
based on the interpretation, adjusting a status of the PFT device to determine a pulmonary function parameter.

2. The PFT system of claim 1, wherein the control system comprises:
an identification module of the PFT device, the identification module comprising a processor communicably coupled with the PFT device and the mouthpiece; and
a controller communicably coupled with the PFT device and the mouthpiece.

3. The PFT system of claim 1, wherein the processor of the identification module is operable to interpret at least one of the unique identifier or the patient data associated with the mouthpiece, and
the controller is operable to adjust the status of the PFT device based on the interpretation to determine a pulmonary function parameter.

4. The PFT system of claim 1, wherein the operation of adjusting a status of the PFT device to determine a pulmonary function parameter comprises enabling the PFT device to determine the pulmonary function parameter.

5. The PFT system of claim 1 wherein the operation of adjusting a status of the PFT device to determine a pulmonary function parameter comprises disabling the PFT device to determine the pulmonary function parameter.

6. The PFT system of claim 1, wherein the control system is further operable to perform operations comprising:
providing a notification to an operator of the PFT device of the disabling of the PFT device to determine the pulmonary function parameter.

7. (canceled)

8. The PFT system of claim 1, wherein the control system is further operable to perform operations comprising:
based on disabling the PFT device to determine the pulmonary function parameter, invalidating an identification marker that comprises at least one of the unique identifier or the patient data.

9. The PFT system of claim 1, wherein invalidating an identification marker that comprises at least one of the unique identifier or the patient data comprises at least one of:
erasing at least one of the unique identifier or the patient data;
changing the unique identifier; or
adjusting a validity flag of the identification module.

10. (canceled)

11. The PFT system of claim 1, wherein the PFT comprises one of a spirometer, a plethysmograph, a diffusion capacity device, a helium dilution device, a nitrogen wash-out device, or an impulse oscillometry device.

12. (canceled)

13. (canceled)

14. The PFT system of claim 1, further comprising a wireless communication module configured to transmit data that comprises at least one of the unique identifier or the patient data between one or more of the mouthpiece, the PFT device, or the control system.

15. The PFT system of claim 1, wherein the wireless communication module comprises:
an RFID tag associated with the mouthpiece, the RFID tag storing the unique identifier and the patient data;
an RFID antenna associated with the mouthpiece to wirelessly transmit signals comprising at least one of the unique identifier or the patient data from the RFID tag; and
an RFID reader associated with the PFT device to identify the mouthpiece based on the signals received from the RFID antenna.

16. The PFT system of claim 1, wherein the RFID antenna comprises a predetermined identification range, and the control system is further operable to perform operations comprising:
based on the mouthpiece and the PFT device being within the predetermined identification range, initiating the interpretation of at least one of the unique identifier or the patient data associated with the mouthpiece.

17. (canceled)

18. (canceled)

19. (canceled)

20. (canceled)

21. (canceled)

22. The PFT system of claim 1, wherein the wireless communication module comprises a first wireless component attached to the mouthpiece and a second wireless component attached to the PFT device, the control system further operable to perform operations comprising:
transmitting the patient data between the first and second wireless components.

23. (canceled)

24. (canceled)

25. (canceled)

26. (canceled)

27. The PFT system of claim 1, wherein the control system is further operable to perform operations comprising:
associating, based on the interpretation of at least one of the unique identifier or the patient data associated with the mouthpiece, the unique identifier with a patient that is uniquely associated with the patient data; and
logging the association of the unique identifier with the patient in a database.

28. (canceled)

29. The PFT system of claim 1, wherein the mouthpiece is a first mouthpiece associated with a first patient, and the control system is further operable to perform operations comprising:
receiving a request to perform a pulmonary test with the PFT device on a second patient;
determining that the second patient is different than the first patient; and
disabling the PFT device based on the determination that the second patient is different than the first patient.

30. The PFT system of claim 1, wherein the control system is further operable to perform operations comprising:
interpreting a second unique identifier and second patient data, associated with a second mouthpiece, that is different that the respective unique identifier and patient data; and
based on the interpretation, enabling the PFT device to determine a pulmonary function parameter of the second patient.

31. The PFT system of claim 1, wherein the control system is further operable to perform operations comprising:
identifying a testing time range associated with at least one of the mouthpiece or the PFT device; and
disabling the PFT device based on the testing time range being greater than a time threshold value.

32. (canceled)

33. The PFT system of claim 1, wherein the mouthpiece further comprises an identification marker that stores permanent data that comprises the unique identifier and semi-permanent data that comprises the patient data.

34. (canceled)

35. (canceled)

36. (canceled)

37. A method for operating a pulmonary function testing (PFT) system, comprising:
providing a PFT system comprising: a PFT device operable to receive a respiratory airflow from a patient through an airflow chamber, a mouthpiece coupled to the PFT device and in airflow communication with the airflow chamber, the mouthpiece comprising a bacterial filter and associated with a unique identifier and patient data, and a control system communicably coupled with the mouthpiece and the PFT device;
interpreting, with the control system, at least one of the unique identifier or the patient data associated with the mouthpiece; and
based on the interpretation, adjusting, with the control system, a status of the PFT device to determine a pulmonary function parameter.

38-71. (canceled)

72. A computer program product encoded on a non-transitory storage medium of a PFT system that comprises a PFT device operable to receive a respiratory airflow from a patient through an airflow chamber, a mouthpiece coupled to the PFT device and in airflow communication with the airflow chamber, the mouthpiece comprising a bacterial filter and associated with a unique identifier and patient data, and a control system communicably coupled with the mouthpiece and the PFT device, the storage medium comprising computer readable instructions for causing one or more processors to perform operations comprising:
interpreting at least one of the unique identifier or the patient data associated with the mouthpiece; and
based on the interpretation, adjusting a status of the PFT device to determine a pulmonary function parameter.

73-106. (canceled)
